(19) United States
(12) Patent Application Publication
(10) Pub. No.: US 2014/0128468 A1
(43) Pub. Date: May 8, 2014

(54) TOPICAL FILM-FORMING COMPOSITION, AND USE THEREOF FOR TREATING MYCOSES

(71) Applicant: LABORATOIRES URGO, Chenove (FR)

(72) Inventors: Claire Bouvier, Dijon (FR); Nathalie Derain, Prenois (FR)

(73) Assignee: LABORATOIRES URGO, Chenove (FR)

(21) Appl. No.: 14/128,114

(22) PCT Filed: Jun. 21, 2012
(86) PCT No.: PCT/FR2012/051420

§ 371 Date: Dec. 20, 2013

(30) Foreign Application Priority Data
Jun. 22, 2011 (FR) 1155492

(51) Int. Cl. A61K 47/44; A61K 45/06; A61K 47/38; A61K 31/20; A61K 31/19

(52) U.S. Cl. CPC A61K 47/44; A61K 31/20; A61K 31/19; A61K 47/38; A61K 45/06
USPC 514/557

(57) ABSTRACT

The present invention relates to a topical film-forming composition for the treating dermatophytosis, specifically mycoses of the feet and, in particular, for treating the symptoms associated with dermatophytosis, specifically the symptoms associated with athlete's foot. The composition is particularly suitable for treating cracking, and for promoting the healing of skin lesions caused by said inflammation. The present composition is also suitable for preventing bacterial or fungal proliferation in the damaged skin.

The subject of the present invention is a topical film-forming composition comprising at least one hydroxamic acid and at least one alpha-hydroxy acid as well as the use thereof for treating dermatophytosis, more specifically mycoses of the feet, and in particular for treating the symptoms associated with dermatophytosis, more specifically the symptoms associated with athlete's foot.

Dermatophytosis is an infection of the skin or skin appendages due to microscopic filamentous fungi: dermatophytes. They belong to 3 genera, Trichophyton, Microsporum and Epidermophyton. These fungi are keratinophilic; they have a predilection for the keratin of the horny layer of the skin, of body hair, of head hair and nails in human beings, and of the skin, of the hair and of the claws in animals. They are responsible for superficial cutaneous infections of the skin and skin appendages, but the mucous membranes are never affected. Dermatophytes are always pathogenic, absent from the permanent or transient commensal flora of the skin. Among the known forms of dermatophytosis, mention may, by way of example, be made of ringworm (which affects more particularly head hair or body hair), epidermophytosis (which affects hairless skin), onyxis and dermatomycosis.

Athlete's foot (from the Latin name tinea pedis) is a dermatophytosis, i.e. a chronic infection of the feet by microscopic fungi.

Athlete's foot constitutes one of the most common skin diseases in the United States after acne, and can affect up to 15% of the population.

Three main fungi are in particular responsible for the inflammation of athlete's foot: Trichophyton, in particular Trichophyton rubrum or Trichophyton mentagrophytes, Epidermophyton, Microsporum and the yeast Candida albicans.

The infection generally manifests itself through pruritic, erythematous or inflamed areas on the feet, in particular between the toes. It can reveal itself through a crack, itching, pieces of white skin which detach, or even liquid-filled blisters. A typical whitish, macerated, split and painful membrane appearance may also be immediately observed.

Without treatment, the infection can propagate to all the spaces between the toes. It can also reach the top and bottom of the foot, where the development of red areas which are very itchy may then be noted. An attack on the nails of the feet (thick, brittle nails) and/or on the sole of the foot (appearing thick, with skin which detaches) may also be observed.

Finally, an infection with bacteria (Staphylococcus aureus or Pseudomonas aeruginosa, for example) may also occur secondarily.

In the simplest cases, athlete's foot is treated locally with compositions in the form of creams, sprays, liquids or powders comprising imidazole-based antifungal compounds, such as clotrimazole or miconazole, zinc undecenoate, or allylamines, such as terbinafine or tolnaflate.

In the most serious cases, or if the infection is resistant to topical treatments, oral antifungals may be prescribed for systemic treatment, alone or as a supplement to topical antifungal compositions. These oral treatments make it possible to treat the inflammation without, however, aiding the healing of the damaged areas of the feet, affected by the infection.

Consequently, there is a constant need for new topical antifungal compositions for effectively locally treating the wounds caused by mycoses of the feet, in particular athlete's foot.

The subject of the present invention is a topical film-forming composition for treating the symptoms associated with dermatophytosis, more specifically the symptoms associated with athlete's foot, and in particular the cracking, and for promoting the healing of the skin lesions caused by this inflammation. The present composition also makes it possible to prevent bacterial or fungal proliferation in the damaged skin.

In particular, the subject of the invention, according to a first aspect, is a topical film-forming composition comprising, in a pharmaceutically acceptable medium, at least one hydroxamic acid, salts thereof and/or complexes thereof, and at least one alpha-hydroxy acid.

For the purposes of the present patent application, the term “topical composition” is intended to mean a form of administration of a composition on a predetermined region of the body.

According to the invention, the topical composition may be a solution, a cream, an ointment, a salve, a gel, a lotion or a varnish.

The film-forming composition according to the invention is preferably water-resistant, i.e. the film that it forms when it is applied to the skin or skin appendages does not decompose in the presence of water.

The invention also relates to the use of such a composition for treating mycoses of the feet and in particular for treating the symptoms associated with athlete's foot.

Contrary to the usual antifungal compositions, such as the Mycoster® product comprising cyclopiroxolamine, the film-forming composition according to the invention makes it possible not only to treat the fungi responsible for mycoses of the feet, but also to effectively inhibit bacterial proliferation at the level of the damaged skin, in particular by filling the cracks caused by these skin infections.

### Hydroxamic Acid

The composition according to the invention comprises at least one hydroxamic acid, salts thereof and/or complexes thereof. These compounds are in particular described in detail in document WO 2009/070736.

In the context of the present application, the hydroxamic acid is preferably an alkyl hydroxamic acid.

The hydroxamic acid, in particular the alkyl hydroxamic acid, can be used in the compositions of the invention in a (non-neutralized) free form or in (neutralized) salt form.

For the purposes of the present invention, the terms “hydroxamic acid” and “alkyl hydroxamic acid” thus include the free forms, the salts or complexes of said acids, and also the precursors thereof, and the salts and complexes of these precursors.

The alkyl hydroxamic acids are compounds of formula (I):

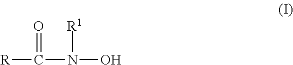

in which R is a substituted or unsubstituted, linear or branched carbon-based chain comprising from two to twenty-two carbon atoms, which chain can be interrupted with one or more oxygen atoms, and can include saturated or unsaturated carbon bonds.

The R groups can include, for example, alkyl, alkenyl, alkynyl, alkoxy, alkenoxy and alkynoxy groups and similar groups, which are linear or branched, and which may be functionalized with substitution groups including hydroxyl groups or other pharmaceutically or cosmetically acceptable functional groups.

R¹ may be hydrogen or the R radical previously described.

The alkyl hydroxamic acid may therefore in particular comprise a substituted or unsubstituted, saturated or unsaturated, linear or branched carbon-based chain comprising from two to twenty-two carbon atoms, and preferably from 6 to 12 carbon atoms.

The carbon-based chains may comprise ethylenic unsaturations, and/or bear particular chemical groups, chosen according to the final properties desired for the alkyl hydroxamic acid.

For example, the presence of hydroxyl groups on the chain may allow better compatibility of the compound with water.

The carbon-based chains may also comprise other pharmaceutically or cosmetically acceptable functional groups.

The alkyl hydroxamic acids may be synthesized from natural oils using lipase catalysis and also other hydroxamic synthesis techniques known to those skilled in the art.

Preferably, the alkyl hydroxamic acid corresponds to formula (I) in which R is a substituted or unsubstituted, saturated or unsaturated, linear or branched carbon-based chain comprising from two to twenty-two carbon atoms, and preferably from 6 to 12 carbon atoms, and R¹ is hydrogen.

The alkyl hydroxamic acid is preferably chosen from the group consisting, in a nonlimiting manner, of hexanohydroxamic acid, caprylohydroxamic acid, caprohydroxamic acid, laurohydroxamic acid, and mixtures thereof.

According to one particularly preferred form of the invention, the alkyl hydroxamic acid is chosen from caprylohydroxamic acid (i.e. having a linear hydrocarbon-based chain comprising eight carbon atoms) and caprohydroxamic acid (i.e. having a linear hydrocarbon-based chain comprising ten carbon atoms).

According to a more preferred embodiment of the invention, the alkyl hydroxamic acid is caprylohydroxamic acid.

It should be noted that the precursors of the alkyl hydroxamic acid, such as hydroxy acids in combination with, for example, hydroxylamine hydrochloride or similar compounds, which can react within the composition of the invention so as to form the alkyl hydroxamic acid, or salts and/or complexes thereof, can be introduced into the composition according to the invention in place of the alkyl hydroxamic acid.

In the context of the present invention, the hydroxamic acid is present in a content ranging from 0.01% to 10% by weight, preferably from 0.1% to 8% and more preferentially from 0.1% to 5% by weight, relative to the total weight of the composition.

According to one particular embodiment, the hydroxamic acid is introduced into the composition according to the invention in the form of a premix with at least one alcohol.

The alcohols that are particularly preferred for forming a premix with the hydroxamic acid are vicinal diols.

The term “vicinal diol” is used when the two hydroxyl groups are in the vicinal position, i.e. attached to adjacent carbon atoms.

By way of example of a vicinal diol usable in the context of the present patent application, mention may be made, in a nonlimiting manner, of ethylene glycol (ethane-1,2-diol), propylene glycol (propane-1,2-diol), 1,2-pentanediol, 1,2-hexanediol, caprylyl glycol, 1,2-decanediol and also glycerol derivatives, such as glyceryl monoethers, for example ethylhexylglycerin, glyceryl monolaurate, glyceryl monocaproate or glyceryl monocaprylate.

The compositions according to the invention preferably comprise vicinal diols in a content ranging from 0.1% to 45% by weight and preferably from 0.5% to 20% by weight, relative to the total weight of the composition.

According to one preferred embodiment, the hydroxamic acid is introduced into the composition according to the invention in the form of a premix with caprylyl glycol and glycerol.

Premixes of hydroxamic acid with caprylyl glycol and glycerol which may be suitable in the context of the present patent application are in particular the products sold by the company Inolex under the name Spectrastat.

### Alpha-Hydroxy Acid

The composition according to the invention also comprises at least one alpha-hydroxy acid. α-Hydroxy acids (or AHAs) are carboxylic acids derived from fruit or milk sugars. The α-hydroxy acids most commonly used are glycolic acid and lactic acid since they have a particular ability to penetrate the skin and to enable the retention of moisturization, and also the initiation of collagen formation.

The alpha-hydroxy acid according to the invention can in particular be chosen from the group consisting of lactic acid, glycolic acid, malic acid, citric acid, tartaric acid, and mixtures thereof.

According to one preferred embodiment, the alpha-hydroxy acid introduced into the composition according to the invention is glycolic acid.

The alpha-hydroxy acid may be present in the composition in a content ranging from 0.01% to 10% by weight and preferably from 0.1% to 5% by weight, relative to the total weight of the composition.

### Pharmaceutically Acceptable Medium

The composition according to the invention comprises a pharmaceutically acceptable medium comprising at least one organic solvent.

For the purposes of the present patent application, the term “pharmaceutically acceptable medium” is intended to mean a medium that is compatible with the skin.

The organic solvent may in particular be chosen from:
ketones that are liquid at ambient temperature, such as methyl ethyl ketone, methyl isobutyl ketone, diisobutyl ketone, isophorone, cyclo-hexanone or acetone;
alcohols, other than the vicinal diols previously described, that are liquid at ambient temperature, such as ethanol, isopropanol, diacetone alcohol, 2-butoxyethanol or cyclohexanol;
propylene glycol ethers that are liquid at ambient temperature, such as propylene glycol monomethyl ether, propylene glycol monomethyl ether acetate or dipropylene glycol mono(n-butyl) ether;
cyclic ethers, such as γ-butyrolactone;
short-chain esters (having from 3 to 8 carbon atoms in total), such as ethyl acetate, butyl acetate, methyl acetate, propyl acetate, isopropyl acetate, isopentyl acetate, methoxy-propyl acetate or butyl lactate;
ethers, such as diethyl ether, dimethyl ether or dichlorodiethyl ether;
alkanes that are liquid at ambient temperature, such as decane, heptane, dodecane or cyclohexane;
alkyl sulfoxides, such as dimethyl sulfoxide;
aldehydes that are liquid at ambient temperature, such as benzaldehyde or acetaldehyde;
ethyl 3-ethoxypropionate;
carbonates, such as propylene carbonate or dimethyl carbonate;
acetals, such as methylal;
and mixtures thereof.

According to one preferred embodiment, the organic solvent is chosen from:
alcohols, other than the vicinal diols previously described, that are liquid at ambient temperature, such as ethanol, isopropanol, diacetone alcohol, 2-butoxyethanol or cyclohexanol, and preferably ethanol,
short-chain esters (having from 3 to 8 carbon atoms in total), such as ethyl acetate, butyl acetate, methyl acetate, propyl acetate, isopropyl acetate, isopentyl acetate, methoxy-propyl acetate or butyl lactate, and preferably ethyl acetate,
   and mixtures thereof.

According to a more preferred embodiment, the organic solvent used in the compositions according to the invention consists of a mixture of ethanol and ethyl acetate.

The organic solvent may represent from 40% to 98% by weight, preferably from 60% to 95% by weight and more preferentially from 70% to 90% by weight, relative to the total weight of the composition.

### Film-Forming Polymer

The composition advantageously comprises at least one film-forming polymer.

According to the present invention, the term “film-forming polymer” is intended to mean a polymer capable of forming, by itself or in the presence of an auxiliary film-forming agent, a continuous and adherent film on a substrate, in particular on the skin.

According to one preferred embodiment, the film-forming polymers used in the composition according to the invention are water-insoluble.

Among the film-forming polymers usable in the composition of the present invention, mention may be made of synthetic polymers, of radical type or of polycondensate type, polymers of natural origin, and mixtures thereof.

The film-forming polymer may be chosen in particular from cellulose-based polymers, such as nitrocellulose, cellulose acetate, cellulose acetate butyrate, cellulose acetate propionate, ethylcellulose, or else polyurethanes, acrylic polymers, vinyl polymers, polyvinyl butyrals, alkyd resins, resins resulting from the products of aldehyde condensation, such as aryl-sulfonamide/formaldehyde resins, for instance toluenesulfonamide/formaldehyde resin, arylsulfonamide/epoxy resins or else ethyl tosylamide resins, or polyvinyl methyl ether/maleic anhydride (PVM/MA) copolymer.

As film-forming polymer, use may in particular be made of the nitrocellulose RS ⅛ sec.; RS [¼] sec.; RS [½] sec.; RS 5 sec.; RS 15 sec.; RS 35 sec.; RS 75 sec.; RS 150 sec.; AS [¼] sec.; AS [½] sec.; SS [¼] sec.; SS [½] sec.; SS 5 sec., in particular sold by the company Hercules, or the nitrocellulose DHL 120/170 IPA sold by the company Nobel; the toluenesulfonamide/formaldehyde resin Ketjentflex MS80 from the company Akzo or Santolite MHP or Santolite MS 80 from the company Faconnier or Resimpol 80 from the company Pan Americana, the alkyd resin Beckosol ODE 230-70-E from the company Dainippon, the acrylic resin Acryloid B66 from the company Rohm & Haas, the polyurethane resin Trixene PR 4127 from the company Baxenden.

According to one preferred embodiment of the present patent application, the film-forming polymer used in the composition is nitrocellulose, in particular sold by the company Nobel under the name DHL 120/170 IPA.

The film-forming polymer may be present in the composition according to the invention in a dry matter content ranging from 0.1% to 20% by weight and preferably ranging from 1% to 15% by weight, relative to the total weight of the composition.

### Auxiliary Film-Forming Agent

In order to improve the film-forming properties of the composition, an auxiliary film-forming agent may advantageously be added.

The auxiliary film-forming agent is, of course, different than the organic solvent present in the pharmaceutically acceptable medium.

Such an auxiliary film-forming agent may be chosen from all the compounds known to those skilled in the art as being capable of performing the desired function, and may in particular be chosen from plasticizers and coalescence agents for the film-forming polymer(s).

Thus, the composition may also comprise at least one plasticizer and/or one coalescence agent. In particular, mention may be made of, alone or as a mixture, the usual plasticizers and coalescence agents, such as:
fatty alcohols, for instance octyldodecanol, 2-butyloctanol, 2-hexyldecanol, 2-undecylpenta-decanol or oleyl alcohol;
glycols and derivatives thereof, such as glycerol, diethylene glycol ethyl ether, diethylene glycol methyl ether, diethylene glycol butyl ether or else diethylene glycol hexyl ether, ethylene glycol ethyl ether, ethylene glycol butyl ether or ethylene glycol hexyl ether;
fatty acids, such as oleic acid, linoleic acid or linolenic acid;
glycol esters, such as triacetin (or glyceryl triacetate);
propylene glycol derivatives, and in particular propylene glycol phenyl ether, propylene glycol diacetate, dipropylene glycol ethyl ether, tripropylene glycol methyl ether and propylene glycol butyl ether;
acid esters, in particular carboxylic acid esters, such as citrates, phthalates, adipates, carbonates, tartrates, phosphates, sebacates and in particular monocarboxylic acid esters, such as isononyl isononanoate, oleyl erucate or 2-octyldodecyl neopentanoate;
oxyethylenated derivatives, such as oxyethylenated oils, in particular vegetable oils, such as sesame oil, castor oil, almond oil, canola oil, hazelnut oil, pistachio oil, linseed oil, borage oil, hemp oil, jojoba oil, sunflower oil, wheat germ oil, corn and/or corn germ oil, peanut oil, avocado oil, safflower oil, rapeseed oil, olive oil, argan oil, sunflower oil, grapeseed oil, soybean oil, walnut oil, marrow seed oil, palm oil, coconut oil, and mixtures thereof. The oil may also be a derivative of one of the vegetable oils mentioned above. It may be hydrogenated or non-hydrogenated and peroxidized or nonperoxidized oil;
   and mixtures thereof.

According to one preferred embodiment, the auxiliary film-forming agent is chosen from oxyethylenated derivatives, such as oxyethylenated oils, in particular vegetable oils, such as castor oil.

According to a more preferred embodiment, the auxiliary film-forming agent is castor oil.

For example, the content of auxiliary film-forming agent may range from 0.01% to 20% and in particular from 0.5% to 15% by weight, relative to the total weight of the composition.

### Additives

The composition according to the invention may comprise one or more pharmaceutically acceptable additives, for instance fragrances, flavorings, dyes, pigments, matting agents, rheological agents, preservatives, vitamins, essential oils and active agents, in particular chosen from antibacterial agents, antiseptics, antivirals, antifungal agents, painkillers, anti-inflammatories, agents for promoting healing, moisturizing agents, depigmenting agents, keratolytic agents, restructuring active agents, anesthetics and sunscreens.

In particular, the active agents which may be introduced into the composition according to the invention may be chosen from:
antibacterials, such as polymyxin B, penicillins (amoxicillin), clavulanic acid, tetracyclines, minocycline, chlortetracycline, aminoglycosides, amikacin, gentamicin, neomycin, silver and salts thereof (silver sulfadiazine), or probiotics;
antiseptics, such as thimerosal, eosin, chlorhexidine, phenylmercuric borate, aqueous hydrogen peroxide solution, Dakin's solution, triclosan, biguanide, hexamidine, thymol, Lugol's solution, iodinated povidone, merbromin, benzalkonium chloride, benzethonium chloride, ethanol or isopropanol;
antivirals, such as aciclovir, famciclovir or ritonavir;
antifungals, such as polyenes, nystatin, amphotericin B, natamycin, imidazoles (miconazole, ketoconazole, clotrimazole, econazole, bifonazole, butoconazole, fenticonazole, isoconazole, oxiconazole, sertaconazole, sulconazole, thiabendazole, tioconazole), triazoles (fluconazole, itraconazole, ravuconazole, posaconazole, voriconazole), allylamines, terbinafine, amorolfine, naftifine or butenafine;
flucytosine (antimetabolite), griseofulvin, caspofungin or micafungin;
painkillers, such as paracetamol, codeine, dextropropoxyphene, tramadol, morphine and derivatives thereof, corticoids and derivatives;
anti-inflammatories, such as glucocorticoids, nonsteroidal anti-inflammatories, aspirin, ibuprofen, ketoprofen, flurbiprofen, diclofenac, aceclofenac, ketorolac, meloxicam, piroxicam, tenoxicam, naproxen, indomethacin, naproxcinod, nimesulide, celecoxib, etoricoxib, parecoxib, rofecoxib, valdecoxib, phenyl-butazone, niflumic acid or mefenamic acid;
active agents for promoting healing, such as retinol, vitamin A, vitamin E, N-acetylhydroxy-proline, Centella asiatica extracts, papain, silicones, essential oils of thyme, of niaouli, of rosemary and of sage, hyaluronic acid, synthetic polysulfated oligosaccharides having 1 to 4 monosaccharide units, such as the potassium salt of sucrose octasulfate, the silver salt of sucrose octasulfate or sucralfate, or allantoin;
moisturizing agents, such as hyaluronic acid, urea, glycerol, fatty acids, modulators of aquaporins, vegetable oils, chitosan, certain sugars, including sorbitol, butters and waxes;
depigmenting agents, such as kojic acid (Kojic Acid SL®—Quimasso (Sino Lion)), arbutin (Olevatin®—Quimasso (Sino Lion)), the mixture of sodium palmitoylproline and European waterlily extract (Sepicalm®—Seppic), undecylenoyl phenylalanine (Sepiwhite®—Seppic), the liquorice extract obtained by fermentation of Aspergillus and ethoxydiglycol (Gatuline Whitening®—Gaffefossé), octadecenedioic acid (ODA White®—Sederma), alpha-arbutin (Alpha-arbutin®, SACI-CFPA (Pentapharm)), the aqueous extract of Arctophylos uva-ursi leaves (Melfade-J®—SACI-CFPA (Pentapharm)), the complex plant mixture Gigawhite® (SACI-CFPA (Alpaflor)), diacetylboldin (Lumiskin®—Sederma), satsuma extract (Melaslow®—Sederma), the mixture of lemon extract enriched in citric acid and of cucumber extract (Uninontan® U-34—Unipex), the mixture of Rumex occidentalis extract and of vitamin C (Tyrostat® 11—Unipex), oligopeptides (Melanostatin 5®—Unipex), kojic dipalmitate (KAD-15®—Quimasso (Sino Lion)), the complex of natural origin Vegewhite® from LCW, wheat germ extracts (Clariskin®—II Silab) or ethylenediaminetriacetate (EDTA);
keratolytic agents, such as salicylic acid, zinc salicylate, ascorbic acid, alpha-hydroxy acids (glycolic, lactic, malic, citric or tartaric acid), silver maple, sour cherry or tamarind extracts, urea, the topical retinoid Keratoline® (Sederma), proteases obtained by fermentation of Bacillus subtilis, the product Linked-Papain® (SACI-CFPA) or papain (proteolytic enzyme derived from the papaya fruit);
restructuring active agents (for example, restructuring active agents for skin appendages), such as silica derivatives, vitamin E, camomile, calcium, horsetail extract or silk lipester;
anesthetics, such as benzocaine, lidocaine, dibucaine, pramoxine hydrochloride, bupivacaine, mepivacaine, prilocaine or etidocaine;
sunscreens, such as chemical screening agents (oxybenzone, sulisobenzone, dioxybenzone, Tinosorb S®, avobenzone, 2-ethoxyethyl p-methoxycinnamate, Uvinul® A+, Mexoryl® XL, octyl methoxycinnamate or octinoxate, octyl salicylate or octisalate, octyl triazine or Uvinul® T 150, methyl salicylate, meradimate, enzacamene, MBBT or Tinosorb® M, octyl cyano-phenylcinnamate or Parsol® 340, para-aminobenzoic acid, ensulizole, Parsol® SLX or polysiloxane-15 or benzylidene malonate polysiloxane, triethanolamine salicylate or trolamine salicylate, Mexoryl® SX or terephthalylidene dicamphorsulfonic acid) and inorganic screening agents (zinc oxides, titanium dioxide, kaolin, ichthyol).

### Use of the Composition

According to one particular embodiment, the subject of the invention is the composition as previously defined, for use in a method for treating dermatophytosis, more specifically mycoses of the feet, and in particular for treating the symptoms associated with dermatophytosis, more specifically the symptoms associated with athlete's foot.

According to another embodiment, the subject of the invention is also the composition as previously defined, for the use in a method for healing wounds caused by dermatophytosis, and in particular by athlete's foot.

In particular, in the context of these uses, the composition according to the invention also makes it possible to prevent bacterial and fungal proliferations at the level of the skin ailment.

The present invention is illustrated in greater detail in the nonlimiting examples described hereinafter.

### EXAMPLE 1

A topical film-forming composition according to the invention having the following composition was prepared:

| Composition | % by weight |
|---|---|
| Nitrocellulose DHL 120/170 IPA sold | 6.00 |
| by the company Nobel | |
| Castor oil (Ricinus communis | 4.70 |
| (castor) seed oil) sold by the | |
| company Prod'hyg Laboratoire | |
| Ethanol sold by the company | 27.77 |
| Charbonneau Brabant | |
| Ethyl acetate sold by the company | 55.53 |
| Darfeuille | |
| Mixture of caprylohydroxamic acid, | 5.00 |
| caprylyl glycol and glycerol | |
| (Spectrastat sold by the company | |
| Inolex) | |
| Glycolic acid sold by the company | 1.00 |
| Dupont | |

All the ingredients, with the exception of the nitro-cellulose, were mixed with stirring for 10 minutes with a propeller stirrer.

The nitrocellulose is then dispersed in the mixture with stirring.

A placebo topical film-forming composition having the following composition was prepared:

| Composition | % by weight |
|---|---|
| Nitrocellulose DHL 120/170 IPA sold | 6.00 |
| by the company Nobel | |
| Castor oil (Ricinus communis | 5.42 |
| (castor) seed oil) sold by the | |
| company Prod'hyg Laboratoire | |
| Ethanol sold by the company | 29.53 |
| Charbonneau Brabant | |
| Ethyl acetate sold by the company | 59.05 |
| Darfeuille | |

All the ingredients, with the exception of the nitro-cellulose, were mixed with stirring for 10 minutes with a propeller stirrer.

The nitrocellulose is then dispersed in the mixture with stirring.

The film-forming composition according to the invention is brought into contact with a known inoculum of dermatophyte Trichophyton mentagrophytes ATCC9533 and the fungal activity of the composition according to the invention was quantitatively evaluated after 7 days of contact according to standard NF EN 1275: Chemical disinfectants antiseptics and: quantitative suspension test for assessing the basic fungicidal and yeasticidal activity of chemical disinfectants antiseptics and—Test method and prescriptions (phase 1)—April 2006.

i. The Culture Media and the Diluent Used are the Following:
Sabouraud agar:

Saboraud agar:

| | | |
|---|---|---|
| Mycological peptone | 10.0 | g |
| Glucose monohydrate | 40.0 | g |
| Agar | 15.0 | g |
| Purified water | 1000 | ml |

Sabouraud broth:

| | | |
|---|---|---|
| Acid hydrolysate of casein | 5.0 | g |
| Meat peptone | 5.0 | g |
| Glucose monohydrate | 40.0 | g |
| Mycological peptone | 20.0 | g |
| Distilled water | 1000 | ml |

Tryptone salt solution (NF EN 1275 - April 2006)

| | | |
|---|---|---|
| Sodium chloride | 9.0 | g |
| Peptone | 1.0 | g |
| Distilled water | 1000.0 | ml |

Sterilized by autoclaving (121° C. for 15 minutes).

ii. Preparation of the Suspension of Trichophyton mentagrqphytes
A Roux flask containing Sabouraud agar medium is inoculated with the microorganism Trichophyton mentagrophytes and incubated at 30° C.±1° C. until spores are obtained (9 to 11 days).
10 ml of sterile physiological saline+0.05% Tween are introduced per Roux flask.
The surface is scraped using a sterile Pasteur pipette converted into a scraper.
The resulting suspension is recovered and filtered through a cell sieve with a 40 μm diameter.
The fungal suspension is prepared in Sabouraud broth and adjusted so as to be at approximately 10⁷ CFU (colony-forming units)/ml. This inoculum is denoted N. The exact titer of the inoculum is determined by agar plate counting, by depositing 2×1 ml of the 10⁻⁵ and 10⁻⁶ dilutions prepared in tryptone salt solution and by adding 15 to 20 ml of molten Sabouraud agar and by then incubating for 48 to 72 hours at 30° C.
   iii. The Tests were Carried Out According to the Following Method:
1 ml of Trichophyton mentagrophytes suspension was deposited on the film-forming composition contained in a Petri dish 90 mm in diameter.
9 ml of Sabouraud broth were added.
This was left to incubate at 37° C.±1° C. for 24 h.
1 ml of the solution obtained was removed and a series of dilutions in Sabouraud broth was carried out down to the 10⁻⁶ dilution.
1 ml of the 10⁻¹ to 10⁻⁶ dilutions was deposited in duplicate and molten Sabouraud agar at 47° C. was added.
This was left to incubate at 30° C. for 7 days.
   iv. Results:

| Composition tested | Number of spores/ml |
|---|---|
| Placebo | 6.0 × 10⁵ |
| Composition according | 0 |
| to the invention | |

The resulting topical film-forming composition therefore makes it possible to prevent fungal proliferation. Thus, the composition according to the invention enables the treatment of mycoses of the feet and in particular the treatment of the symptoms associated with athlete's foot.

### EXAMPLE 2

A topical formulation having the following composition was prepared:

| Composition | Weight in g |
|---|---|
| Castor oil (Ricinus communis | 4.18 |
| (castor) seed oil) sold by the | |
| company Prod'hyg Laboratoire | |
| Mixture of caprylohydroxamic | 4.45 |
| acid, caprylyl glycol and | |
| glycerol (Spectrastat sold by the | |
| company Inolex) | |
| Ethanol sold by the company | 24.71 |
| Charbonneau Brabant | |
| Glycolic acid sold by the company | 0.98 |
| Dupont | |
| Water | Qs 100 ml |

All the ingredients were mixed with stirring for 10 minutes with a propeller stirrer.

The composition according to the invention was artificially contaminated by means of an inoculum of microorganisms responsible for mycoses of the feet and in particular for the symptoms associated with athlete's foot. The inoculated preparations were then maintained at a temperature of 22.5±2.5° C. in the dark, and then samples of the medium were taken at given time intervals. The organisms in the samples thus taken were counted.

The tests are carried out in use simulation, that is to say after reinoculations at times T2 days, T7 days and T10 days.

i. The Culture Media Used are the Following:
Sabouraud agar for the fungal strains (European Pharmacopoeia, 7th edition, chapter 2.6.12.).
Trypticase soy agar for the bacterial strains (European Pharmacopoeia, 7th edition, chapter 2.6.12.).
   ii. Preparation of the Microorganism Suspensions:

Bacterial and Yeast Suspensions
The surface of an agar is inoculated with the stock culture recently obtained from each of the micro-organisms specified.
The bacterial cultures are incubated at a temperature of 32.5° C.±2.5° C. for 18/24 h, and the Candida albicans culture at a temperature of 22.5° C.±2.5° C. for 48 h.
The microbial suspensions are prepared and adjusted so as to have a titer of 10⁸ CFU (colony-forming units) per milliliter.
The exact titer of the inoculum is determined by agar plate counting.

The method by agar plate counting at the 10⁻² dilution of the product was validated for the microorganisms tested: Staphylococcus aureus, Pseudomonas aeruginosa, Candida albicans and Trichophyton rubrum.

iii. The Tests were Carried Out According to the Following Method:
A series of containers containing the composition according to the invention was inoculated with a suspension of one of the test microorganisms in order to obtain an inoculum of 10⁵ to 10⁶ micro-organisms per gram of preparation.
To inoculate the product, 100 μl of bacterial suspension having a titer of approximately 10⁸ CFU/ml were introduced into 10 ml of product.
In order to be sure of a homogeneous distribution, the containers are mixed by manual and mechanical (vortex type) stirring.
The inoculated product is then maintained at a temperature of 22.5° C.±2.5° C. in the dark.
1 milliliter is taken from the appropriate samples of each container, at times zero, 2 d, 7 d, 10 d and 14 d.
The number of viable microorganisms is determined for each time by agar plate counting according to the method previously validated.
After each count, the test microorganism is reinoculated into the product at times 2 d, 7 d and 10 d.
At the end of the tests, for each microorganism, the logarithmic reduction in the number of viable microorganisms relative to the value obtained for the inoculum is calculated.
   iv. Results:

Logarithmic reduction in the number of
viable microorganisms relative to the
value obtained for the inoculum

| Strains | T: 2 d | T: 7 d | T: 10 d | T: 14 d |
|---|---|---|---|---|
| Staphylococcus | >4.5 | >4.5 | >4.5 | >4.5 |
| aureus | | | | |
| Pseudomonas | >4.4 | >4.4 | >4.4 | >4.4 |
| aeruginosa | | | | |
| Candida | >4.6 | >4.6 | >4.6 | >4.6 |
| albicans | | | | |
| Trichophyton | >3.4 | >3.4 | >3.4 | >3.4 |
| rubrum | | | | |

It was thus demonstrated that the compositions according to the invention thus make it possible not only to treat the fungi responsible for mycoses of the feet, but also to effectively inhibit the bacterial proliferation associated with these diseases.

1. A topical film-forming composition comprising, in a pharmaceutically acceptable medium, at least one hydroxamic acid, salts thereof and/or complexes thereof, and at least one alpha-hydroxy acid.

2. The composition as claimed in claim 1, wherein the hydroxamic acid is an alkyl hydroxamic acid.

3. The composition as claimed in claim 2, wherein the alkyl hydroxamic acid comprises a substituted or unsubstituted, saturated or unsaturated, linear or branched carbon-based chain comprising from two to twenty-two carbon atoms.

4-16. (canceled)

17. The composition as claimed in claim 3, wherein the alkyl hydroxamic acid comprises from 6 to 12 carbon atoms.

18. The composition as claimed in claim 2, wherein the alkyl hydroxamic acid is selected from the group consisting of hexanohydroxamic acid, caprylohydroxamic acid, caprohydroxamic acid, laurohydroxamic acid, and mixtures thereof.

19. The composition as claimed in claim 18, wherein the alkyl hydroxamic acid is caprylohydroxamic acid.

20. The composition as claimed in claim 1, wherein the hydroxamic acid is present in a content ranging from 0.01% to 10% by weight, relative to the total weight of the composition.

21. The composition as claimed in claim 1, wherein the hydroxamic acid is present in a content ranging from 0.1% to 5% by weight, relative to the total weight of the composition.

22. The composition as claimed in claim 1, wherein the alpha-hydroxy acid is selected from the group consisting of lactic acid, glycolic acid, malic acid, citric acid, tartaric acid, and mixtures thereof.

23. The composition as claimed in claim 1, wherein the alpha-hydroxy acid is glycolic acid.

24. The composition as claimed in claim 1, wherein the alpha-hydroxy acid is present in a content ranging from 0.01% to 10% by weight, relative to the total weight of the composition.

25. The composition as claimed in claim 1, wherein the alpha-hydroxy acid is present in a content ranging from 0.1% to 5% by weight, relative to the total weight of the composition.

26. The composition as claimed in claim 1, wherein the pharmaceutically acceptable medium comprises at least one organic solvent selected from:
alcohols that are liquid at ambient temperature, such as ethanol, isopropanol, diacetone alcohol, 2-butoxyethanol or cyclohexanol,
short-chain esters (having from 3 to 8 carbon atoms in total), such as ethyl acetate, butyl acetate, methyl acetate, propyl acetate, isopropyl acetate, isopentyl acetate, methoxypropyl acetate or butyl lactate,
and mixtures thereof.

27. The composition as claimed in claim 26, wherein the alcohol is selected from monoalcohols, glycols, and mixtures thereof.

28. The composition as claimed in claim 27, wherein the monoalcohol is ethanol, and the glycol is caprylyl glycol.

29. The composition as claimed in claim 26, wherein the organic solvent consists of a mixture of ethanol and ethyl acetate.

30. The composition as claimed in claim 26, wherein the organic solvent represents from 70% to 90% by weight, relative to the total weight of the composition.

31. The composition as claimed in claim 1, wherein it comprises a film-forming polymer of nitrocellulose.

32. The composition as claimed in claim 1, comprising an auxiliary film-forming agent selected from oxyethylenated vegetable oils.

33. The composition as claimed in claim 32, wherein the oxyethylenated vegetable oil is castor oil.

34. The composition as claimed in claim 1, wherein it also comprises an ingredient selected from antibacterial agents, antiseptics, antivirals, antifungal agents, painkillers, anti-inflammatories, agents for promoting healing, moisturizing agents, depigmenting agents, keratolytic agents, restructuring active agents, anesthetics, sunscreens, and mixtures thereof.

35. Method for treating dermatophytosis, mycoses of the feet, the symptoms associated with dermatophytosis, and/or the symptoms associated with athlete's foot comprising topically administering a composition as claimed in claim 1.
